(19) United States
(12) Patent Application Publication
(10) Pub. No.: US 2009/0048508 A1
(43) Pub. Date: Feb. 19, 2009

(54) Rf coil assembly for use in magnetic resonance imaging

(75) Inventors: Steven Streatfield Gill, Bristol (GB); Mathew David Frederick Stratton, Bristol (GB)

Correspondence Address: OLIFF & BERRIDGE, PLC, P.O. BOX 320850, ALEXANDRIA, VA, 22320-4850, US

(73) Assignee: RENISHAW PLC, WOTTON-UNDER-EDGE (GB)

(21) Appl. No.: 11/921,080

(22) PCT Filed: Jun. 14, 2006
(86) PCT No.: PCT/GB2006/002179

§ 371 Date: Nov. 27, 2007

(51) Int. Cl. G01R 33/32; A61B 5/055

(52) U.S. Cl. 600/422; 324/318

(57) ABSTRACT

An RF coil assembly for use with magnetic resonance imaging (MRI) apparatus is described. The RF coil assembly comprises one or more coil elements capable of receiving electromagnetic radiation and is capable of being directly attached to a stereotactic base ring. The RF coil assembly may comprise a localiser box having fiducial markers. A corresponding attachment apparatus is also described for optimising the attachment position of a stereotactic base ring to a subject's head. The attachment apparatus comprises one or more fixing elements that allow the attachment apparatus to be releasably attached to a stereotactic base ring. In one embodiment, the internal dimensions of the attachment apparatus are selected to be substantially the same as the internal dimensions of an RF coil assembly. Corresponding methods are also described.

The present invention relates to apparatus for use in for magnetic resonance imaging (MRI). In particular, the invention relates to an RF coil assembly for magnetic resonance imaging (MRI) of a subject prior to stereotactic neurosurgery. A method of using such an RF coil assembly and an associated method and apparatus for attaching a stereotactic base ring to a subject are also described.

The success of stereotactic functional neurosurgery is very dependent on the accuracy with which an electrode or catheter can be inserted into a target site of the brain. Structures in the brain can be targeted directly if a radiological imager, for example such as a magnetic resonance (MR) imager, has sufficient sensitivity to visualise the brain with acceptable spatial resolution. If the target cannot be visualised then its likely position can be inferred using an indirect method in which an atlas of the brain can be co-registered with visible landmarks in the brain. Typically, the anterior commisure (AC) and the posterior commisure (PC) in the third ventricle are used as internal landmarks for co-registration to an atlas because they are visible using either ventriculography, computerised tomography scanning or MRI. The use of a standardised brain atlas, even if it is morphed to an individual patient, will not produce the required accuracy because all brains are anatomically unique.

To compensate for individual anatomical variation surgeons using the indirect method of target localisation use interoperative clinical and electrophysical monitoring procedures with the patient awake on the operating table. A commonly used method is to pass one or more microelectrodes through the brain to the presumed vicinity of the target until its characteristic neural firing patterns are recorded. To identify a target in this way usually involves 3 to 6 recording tracts being made to delineate the target's boundaries. This necessarily increases the risk of brain trauma and haemorrhage. In addition, the accuracy with which a target can be defined in the axial plane depends upon the distances between the recording trajectories, which is typically 2 mm. This is the minimum distance apart that a second probe can be inserted without entering the track of the first probe. Therefore the maximal spatial resolution of this technique is 2 mm which is not sufficiently precise to ensure optimal placement of an instrument in some small subcortical brain targets. With properly acquired MRI images taken under strict stereotactic conditions it is possible to target neurosurgical features more accurately.

Due to spatial distortions created by varying magnetic susceptibility of different tissues and matter to be scanned, MRI images taken under strict stereotactic conditions must be correctly aligned to the patient's head by the use of fiducial markers. The fiducial markers are usually arranged in an assembly known as a localizer box which is mounted to the base ring of a stereotactic frame. An RF (radio frequency) coil, such as a birdcage coil, is then placed loosely around the localizer box when capturing an MR image. After the MR image has been taken, the localiser box can be detached from the base ring and stereoguide apparatus can then be attached thereto. The base ring thus provides a fixed reference position during both the image acquisition and treatment processes. In other words, the acquired MR images include fiducial markings having a known spatial relationship to the base ring and the stereoguide also allows surgical implements to be accurately positioned relative to the base ring.

In the above arrangement, the proximity of the RF coil to the patient's head during MR image acquisition is restricted by the localiser box and the stereotactic base ring. This can introduce noise and distortion to the acquired MR images. The object of the present invention is thus to mitigate at least some of the above mentioned disadvantages associated with apparatus for obtaining MR images.

According to a first aspect of the present invention, an RF coil assembly for use with magnetic resonance imaging apparatus is provided, the RF coil assembly comprising one or more coil elements capable of receiving electromagnetic radiation, wherein the RF coil assembly is capable of being directly attached to a stereotactic base ring.

The present invention thus provides an RF coil assembly having at least one coil element, said RF coil assembly being directly attachable to a stereotactic base ring. The term “stereotactic base ring” as used herein would be understood by those skilled in the art to mean the part of a stereotactic frame that is attached, via suitable fixing elements, to a subject. The subject may be an animal or a human. The stereotactic frame can then be used as the basis for locating targets within a three-dimensional space. Stereotactic frames having stereotactic base rings are described in more detail elsewhere; e.g. see “Stereotactic and Functional Neurosurgery/The Practice of Neurosurgery Part XI/Editors G. T. Tindall, P. R. Cooper, D. L. Barrow: Williams & Wilkins 1996”, the contents of which are hereby incorporated herein by reference. The term “coil element” as used herein would also be understood by a person skilled in the art as referring to any antenna-like structure capable of absorbing or radiating electromagnetic radiation. More specifically the term “coil element” refers to an antenna-like structure which resonates or efficiently stores energy at the Larmor frequency and which is composed of inductive elements and capacitive elements.

An RF coil assembly of the present invention has a number of advantages compared with prior art RF coil apparatus of the type described above. For example, providing an RF coil assembly of the present invention allows the distance between the coil elements and the subject's head to be minimised. The RF coil assembly thus permits the coil elements to be placed as close to the head as possible (although direct contact with the skin should be avoided). This should be contrasted to prior RF coil structures of the type described above in which coil elements are located around a stereotactic frame during MRI image acquisition; such prior art arrangements result in MR images being acquired using coil elements that are typically spaced a considerable, and non-repeatable, distance from a subject's head.

Placing coil elements closer to the head in accordance with the present invention allows the signal to noise ratio of MR images to be increased thereby providing clearer, higher resolution, MR images and/or permitting shorter acquisition times.

Neurosurgeons and radiosurgeons can exploit such sharper, higher resolution, images for more accurate and safer surgical procedures, in particular stereotactic procedures. The ability to obtain sharp, high resolution and undistorted images is particularly invaluable in planning surgery in eloquent areas and in localising epileptic foci and functional abnormalities for targeted treatment. Such surgery includes the implantation of electrodes to provide deep brain stimulation, targeted drug therapies and targeted radiotherapy.

The RF coil assembly may advantageously comprise at least one coil element capable of transmitting electromagnetic radiation. The one or more coils capable of receiving electromagnetic radiation may be one or more receiver coils or receiver/transmitter coils, or a combination of such coils. Preferably, the one or more coils of the RF coil assembly are receiver coils. The RF coil assembly may thus comprise at least one of a receiver coil (i.e. a coil used solely to receive electromagnetic energy), a receiver/transmitter coil (i.e. a coil used for both receiving and transmitting electromagnetic energy) and a transmitter coil (i.e. a coil used solely to transmit electromagnetic radiation).

There have been many advances in the design of RF coils in recent years. As well as increasing the number of coil elements, their configuration and the processing of the data that they allow has improved the signal to noise ratio and reduced MRI scan time. The RF coil assembly described herein can be used with any currently available coil technology such as quadrature, multi-element phased array, parallel receive and parallel transmit coil structures. The number and arrangement of coil elements chosen will therefore depend on the requirements of the system.

The one or more coil elements may be arranged within the RF coil assembly in any suitable manner. For example, a single receiver coil element may be arranged as a bird cage receiver coil. Alternatively, a plurality of coil elements can be used. For example. the RF coil assembly could conveniently comprise 2, 4, 6, 8, 16, 32, 64 or 128 or more coil elements. Preferably, 2 to 32 coil elements may be provided. More preferably, 4 to 16 coil elements may be provided. More preferably, 4 to 8 or 8 to 16 coil elements may be provided. Advantageously, the coil elements may be arranged as a multi-element phased array receiver coil assembly. Conveniently, there may be 4, 6, or 8 coil elements in the multi-element phased array coil assembly. Advantageously, there are 4 coil elements in the phased array. There may conveniently be 6 coil elements in the phased array. If a plurality of coil element are provided, the coil elements may advantageously overlap. Preferably the coils are arranged for maximum signal to noise, phased array, parallel imaging or a combination of the above.

Advantageously, the one or more coil elements of the RF coil assembly are connected to low input impedance preamplifiers. Receiver electronics may also be provided for processing signals from the one or more coil elements. Such preamplifiers and/or receiver electronics may form an integral part of the RF coil assembly; e.g. such component may conveniently be located inside the RF coil assembly. Advantageously, the outputs of the low input impedance preamplifiers are connected to a termination box which may be physically mounted to the RF coil assembly (e.g. mounted on the outside of the RF coil assembly). Preferably the termination box is also connected to a flexible cable which may link the termination box to the MRI apparatus optionally through an adapter box. This arrangement means that the flexible cable will not interfere with the one or more receiver coils during use and that any signal losses in the cable are minimised. This arrangement also means that the received signals are amplified, interference and coupling between coils is reduced, and these coils may be used in parallel imaging.

Advantageously, the RF coil assembly comprises a coil support structure to which said one or more coil elements are attached or formed, wherein said coil support structure is capable of being directly attached to a stereotactic base ring. Preferably, the one or more coil elements are rigidly fixed to the coil support structure so that there is substantially no movement between the coil elements and the base ring. If provided, the at least one coil element capable of transmitting electromagnetic radiation may also be attached to that coil support structure. Preferably, the coil support structure is made of a rigid, electromagnetically inert material. The electromagnetically inert material may comprise epoxy resins comprising electromagnetically inert fibres such as glass fibres. Such materials are MRI compatible.

Advantageously, the RF coil assembly comprises a fiducial marker unit. The fiducial marker unit may be in the form of a localiser box or it may comprise a plurality of fiducial marker unit components. The fiducial marker unit (or fiducial marker unit components) may be removably attached to the rest of the RF coil assembly via an attachment mechanism or may be provided integrally with the RF coil assembly. Preferably the fiducial marker unit is securely fixed to the RF coil assembly; e.g. using any means such as bonding using an adhesive or fastened using nuts and bolts, screws, etc. If the fiducial marker unit is to be removably attached to the RF coil assembly (or the stereotactic base ring) the preferred attachment mechanism will allow repeatable repositioning of the fiducial marker unit relative to the RF coil assembly (or the stereotactic base ring); e.g. a kinematic mount may be provided. A preferable mechanism would comprise mutually engageable locating elements on the mating surfaces.

The fiducial marker unit may comprise one or more fiducial markers. Preferably, the fiducial marker unit can be repeatably positioned relative to the stereotactic base ring whilst minimising the distance between the coil elements of the RF coil assembly and the volume to be imaged. As those skilled in the art will appreciate, the fiducial markers in the fiducial marker unit will give an MR signal such that they give a clear reference indicator in each image taken of the brain.

Standard fiducial markers are comprised by material which provide an MR signature (such as tubes filled with an oil or copper sulphate solution) that are arranged to define a square with two opposite edges joined along a diagonal. The described fiducial markers will show up on a MRI slice as sets of three marks such as spots. It is known to have more than one set of fiducial markers arranged in a localizer box so that MRI slices taken in at least the axial plane (and possibly also the coronal and/or sagittal planes) can be geometrically corrected. As the absolute and relative positions of these markers are known, geometric corrections can be applied to ensure that the markers representing the two opposite edges appear in the correct absolute position and that the marker representing the diagonal gives a height or z position that corresponds with what is expected.

A number of localizer boxes are known to those skilled in the art, such as the Leksell system (Elekta Instrument AB, Sweden) and CRW system (Radionics Inc. Burlington Mass.). Such localizer boxes include a fiducial system that can fit closely around the object to be imaged. Such a localiser box may thus be attached to, or integrated with, the RF coil assembly described herein.

The one or more fiducial markers of the fiducial marker unit may be positioned on the inside and/or the outside of the coil elements of the RF coil assembly. The one or more coil elements are preferably positioned within the fiducial markers of the fiducial marker unit; i.e. so that the fiducial marker unit of the RF coil assembly enclose said one or more coil elements. Preferably, the at least one fiducial marker is positioned such that the distance between the one or more coil elements of the RF coil assembly and the object to be imaged is minimised.

Advantageously, the one or more coil elements are arranged to optimise visualisation of the brain fiducial markers and are tuned to maximise signal-to-noise ratio at the centre of an imaging volume. In such an arrangement, the dimensions of the imaging volume may be at least 80 mm laterally, 40 mm antero-posteriorly and 50 mm vertically. Conveniently, the imaging volume encompasses the basal ganglia, thalamus and mid brain.

Advantageously, the one or more coil elements of the RF coil assembly comprise copper. Conveniently, the one or more coil elements are made from ribbons of copper about 5 mm wide. The one or more coil elements are advantageously insulated with any suitable insulating material.

As mentioned above, the RF coil assembly is capable of being directly attached to a stereotactic base ring. The attachment may be via any fixing element or elements that ensure the attachment apparatus is rigidly fixed to the stereotactic base ring. Preferably the mechanism used to attach the RF coil assembly to the stereotactic base ring allows repeatable relative positioning of the two elements; e.g. using a kinematic mount. Advantageously, the RF coil assembly comprises fixing elements, the fixing elements allowing the RF coil assembly to be releasably attached to a stereotactic base ring. Preferably, the fixing elements comprise clips and/or localizer pins. The clips may be arranged to engage with corresponding recesses formed in the base ring and the localizer pins also engage with corresponding holes formed in the stereotactic base ring.

Preferably, the RF coil assembly is shaped to closely fit around the head of an individual. Preferably the RF coil assembly is designed to allow room for the fixation elements used to attach the stereotactic base ring to the subject's head. This could be in the form of a number of gaps so that the posts used to attach the stereotactic base ring to the subject's head can be accommodated by the RF coil assembly when it is attached to the base ring. Preferably the gaps are four longitudinal openings. The RF coil assembly may thus have four sides defining four longitudinal openings, the longitudinal openings being located at the joins of the four sides. Alternatively the coil structure needn't have gaps to accommodate the posts. The coil elements within the RF coil assembly can be arranged so that they are routed around the posts to provide a closely fitting coil structure. The RF coil assembly may have a dimension in a lateral axis of about 200 mm, a dimension in a vertical axis of about 150 mm and a dimension in an anteroposterior axis of about 240 mm.

The RF coil assembly of the present invention may be used with any MRI apparatus. The term “MRI apparatus” means any apparatus that can receive the detected electromagnetic radiation from the one or more coils of the coil assembly and convert the data into an image. Suitable MRI apparatus include Gyroscan Intera 1.5T (Phillips Medical Systems), 3.0 or 1.5T Sigma (General Electric Healthcare) and 1.5T Siemens Magnetom Symphony. Other commercially available MRI systems can also be used with the RF coil assembly. The RF coil assembly is thus preferably compatible with a number of different MRI scanner types. In particular, the one or more coil elements of the RF coil assembly are preferably capable of direct or indirect connection to MRI apparatus, optionally through an adapter box that provides the necessary electronic interface.

The present invention also provides the use of an RF coil assembly of the present invention to obtain a magnetic resonance image of a patient's brain. The present invention also provides a magnetic resonance image obtained by using the RF coil assembly.

As described above, the RF coil assembly of the present invention is preferably fitted to a subject such that the coil elements are located as closely as possible to the head of that subject. This close fitting may be aided by using separate attachment apparatus when attaching the stereotactic base ring to the subject.

The present invention thus also provides attachment apparatus for optimising the attachment position of a stereotactic base ring to a subject's head, the attachment apparatus comprising one or more fixing elements, said one or more fixing elements allowing the attachment apparatus to be releasably attached to a stereotactic base ring, wherein the internal dimensions of the attachment apparatus are substantially the same as the internal dimensions of the above described RF coil assembly.

The attachment apparatus may advantageously comprise one or more markers to enable said attachment apparatus to be aligned with anatomical features of the head. Conveniently, the attachment apparatus is in the form of a helmet which, in use, contacts the crown of the head. Advantageously, the apparatus extends, in use, down the sides of the head. The term “in the form of a helmet” is used broadly herein to mean any rigid structure that can be placed over an individual's head, and rests on the crown of the head and is capable of supporting a stereotactic base ring at a position circumjacent to the individual's head. In this manner, the weight of the attachment apparatus can be supported by the subject's head during alignment; e.g. whilst the one or more markers are aligned with the required anatomical features of the head.

The present invention thus also provides attachment apparatus, optionally in the form of a helmet, that can be releasably attached to a stereotactic base ring. At least one marker may also be provided to permit alignment of the attachment apparatus (and hence alignment of the associated stereotactic base ring) with anatomical features of the subject's head. Once alignment of the stereotactic base ring has been achieved using the attachment apparatus, the stereotactic base ring can be secured directly to the subject and the attachment apparatus removed. The RF coil assembly described above may then be attached to the stereotactic base ring to permit MR imaging of the subject.

The attachment apparatus is preferably sized so that the distance from a part of the attachment apparatus that contacts the crown of the subject's head to the fixing elements is substantially the same as the distance from the top or closed end of the RF coil assembly to the bottom or open end of the RF coil assembly. This ensures that when the RF coil assembly is connected to the base ring, the top or closed end of the RF coil assembly is positioned as close to the crown of the subject's head as possible. In use, the top or closed end of the RF coil structure may be in contact with the crown of the individual's head, provided that the coil elements are not in direct contact with the head.

It is particularly preferred that the attachment apparatus is about 1 to 5 mm shorter than the internal length along the vertical axis (from the open to the closed end) of the RF coil structure. More preferably, the internal length along the vertical axis of the RF coil assembly is preferably about 1 to 2 mm longer than the internal length of the attachment apparatus. This ensures that, on attaching the RF coil assembly to a stereotactic base ring that has been positioned using the attachment apparatus, a small gap (about 1-2 mm or 1-5 mm) exists between the crown of the individual's head and the closed end of the RF coil structure.

Known techniques for attaching a base ring to a subject involve inserting ear bars that are attached to the base ring into the patient's external auditory meati and then pivoting the base ring into the desired alignment. In awake patients this procedure can be painful and it is not uncommon for the ear bars to traumatise the external auditory meati and/or drive wax deep into the patients ear and impair the patients hearing. Furthermore, obtaining appropriate alignment of the stereotactic base ring can be difficult and time consuming.

Attachment apparatus of the present invention permits the stereotactic base ring to be aligned with greater accuracy and less trauma than using ear bar attachment pieces of the type described above. In particular, the attachment apparatus of the present invention allows alignment of the stereotactic base ring with anatomical features of the head which in turn may be used to provide alignment with the required plane within the brain. The attachment apparatus thus allows the position of the stereotactic base ring to be optimised thereby allowing it to be aligned to any desired plane of the brain, e.g., parallel to the individual's orbitomeatal plane. The orbitomeatal plane closely approximates to the orientation of the anterior commisure/posterior commisure plane (AC/PC plane) which is a reference plane used in stereotactic atlases of the brain. When the individual is scanned with images taken parallel to the base ring then they can be directly compared with a brain atlas such as the Schaltenbrand-Bailey atlas described in Introduction to Stereotaxis with an Atlas of the Human Brain/G. Schaltenbrand and P. Bailey/Georg Thieme Verlag/Stuttgart/1959. However, such alignment of the base ring with a plane of the brain is unnecessary if surgical planning software is provided.

Attachment apparatus of this type thus allows the stereotactic base ring to be fixed in the required location on the subject's head without having to use the RF coil assembly itself during the attachment process. As described below, this allows the attachment apparatus to be made from transparent, lightweight material, that may be compatible with sterilisation techniques such as autoclaving. The RF coil assembly may then be affixed to a subject after the stereotactic base ring has been attached using the attachment apparatus.

The present invention thus provides attachment apparatus for optimising the attachment position of a stereotactic base ring to an individual's head, wherein the attachment apparatus may be in the form of a helmet which in use contacts the crown of the head and extends down the sides of the head, and comprises fixing elements enabling it to be fixed to a stereotactic base ring, and markers enabling it to be aligned with anatomical features of the head. As outlined above, although providing the attachment apparatus in the form of such a helmet is preferred, it is by no means essential. In fact, contact of the attachment apparatus with the head could be avoided all together so long as the attachment apparatus can be manoeuvred into alignment relative to the subject's head. For example, the attachment apparatus may be supported by another part of the subject (e.g. it could rest on the subject's shoulders) or by an external support to which the subject may also be attached in some way.

The one or more markers of the attachment apparatus may be any feature(s) on the attachment apparatus that enable alignment with anatomical features of the individual's head. Conveniently, the one or more markers are located on, or formed integrally with, the attachment apparatus. For example, the markers may be moulded or embedded features of the attachment apparatus, or marks formed on the surface of the apparatus. Preferably, the markers are clearly visible. Conveniently, the attachment apparatus is substantially transparent and the markers (e.g. reference lines) are opaque thereby assisting with the alignment of the base ring with anatomical features on the individual. Preferably the markers are in the form of one or more reference lines. Advantageously, a plurality of markers are provided.

Conveniently, the plurality of markers comprises a first set of markers, wherein each marker of said first set of markers comprises a reference line. Advantageously, each line of said first set of markers is substantially parallel to the plane that contains, in use, the stereotactic base ring. Each reference line of said first set may be spaced apart from adjacent lines by a substantially equal distance. The first set of markers may thus comprise a series of equidistantly spaced, substantially parallel, reference lines. These one or more reference lines may be termed “horizontal” reference lines; where horizontal refers to the orientation of the line when the attachment apparatus is attached to an upright (e.g. standing) subject. Providing such a first set of markers allows, in use, alignment of the stereotactic base ring with at least one of the external auditory meati and inferior orbital margins of the subject.

The plurality of markers may conveniently comprise a second set of markers, said second set of markers comprising one or more reference lines that are substantially perpendicular to the plane that contains, in use, the stereotactic base ring. In other words, one or more “vertical” reference lines may be provided. Advantageously, said second set of markers are provided to allow, in use, alignment of the stereotactic base ring with at least one of the antero-posterior and lateral axes of the subject. In this manner, the stereotactic base ring can be orientated along a specific plane.

Preferably, the attachment apparatus is arranged so that the anatomical features of the subject's head can be easily seen. Preferably the attachment apparatus is made from a substantially transparent material. Preferably the substantially transparent material comprises acrylic (e.g. Perspex). Alternatively the attachment apparatus could be constructed from non-transparent materials in a configuration that allows visibility of the anatomical features; e.g. having cut-away sections near the anatomical regions of interest. It should be noted that instead of arranging the attachment apparatus so that visible reference features of the subject's head can be seen, the attachment apparatus may be shaped so as to rest on the subject's head in the required orientation. In this manner, no manual alignment step is required during attachment of the stereotactic base ring.

To maximise visibility of the subject's head, it is preferred that the attachment apparatus performs only the attachment and alignment functions. In other words, the attachment apparatus preferably does not comprise an RF coil nor a fiducial marker. The separate RF coil assembly described above can thus be substituted for the attachment apparatus when the subject is to be imaged using MRI apparatus. Separate stereoguide apparatus may also be attached to the stereotactic base ring for performing a stereotactic surgical procedure.

The attachment apparatus may be attached to the stereotactic base ring via any fixing element or elements that ensure the attachment apparatus is rigidly fixed to the stereotactic base ring. Preferably the mechanism used to attach the attachment apparatus to the stereotactic base ring allows repeatable relative positioning of the two elements. Preferably the attachment apparatus is attached to the stereotactic base ring by one or more fixing elements that comprise one or more clips. Preferably the one or more fixing elements comprise one or more mutually engageable locating elements (e.g. localiser pins) on the mating surfaces of the stereotactic base ring and attachment apparatus to allow repeatable relative positioning. Preferably, the fixing elements that enable the RF coil assembly to be attached to the base ring are identical to those which enable the attachment apparatus to be attached to the base ring. In this manner, the RF coil assembly and attachment apparatus may be attached to the stereotactic base ring.

The present invention also provides a kit that includes at least one of the above described RF coil assembly and attachment apparatus. The kit may also comprise an additional frame portion that attaches to the stereotactic base ring. In other words, the stereotactic base ring may be part of a larger stereotactic frame. The kit may also comprise one or more stereotactic surgical instruments. The kit may also comprise a fiducial marker unit (or localiser box) for use with MRI apparatus. The kit may additionally comprise apparatus for attaching the fiducial marker unit to the RF coil assembly. The fiducial marker unit may be attached or integral with the RF coil assembly or it directly attached to the stereotactic base ring. The kit may also include fixing elements such as screws or adhesive etc as appropriate. The kit may also comprise a phantom to aid correction of geometrical distortion from the MRI scanner, additionally software may be included to calculate the error map for the MRI scanner, the software may also be designed to assist the localising of brain structure targets and planning potential implant trajectories.

The kit advantageously comprises at least one of a stereotactic base ring and a base ring holder. As described above, a stereotactic base ring may be attached to a subject using attachment apparatus of the present invention. The stereotactic base ring may also be arranged to attach to a stereotactic base ring holder. Base ring holders are known and comprise any device which directly or indirectly attaches to an MRI couch and which also attaches to a stereotactic base ring such that the stereotactic base ring is held in a position fixed relative to the MRI couch. The base ring holder can also be used to adjust the position of the base ring relative to the MRI couch, such that, for example, the base ring is parallel to a standard axial image acquisition. In this way the subject may be aligned and fixed in position in the MRI scanner. Immobilising the subject relative to the polarising magnetic field of the MRI apparatus during the scanning process using a base ring holder ensures that the best quality images are obtained. In this manner, the stereotactic base ring is capable of being immobilised relative to the MRI apparatus.

The stereotactic base ring can be fixed to the subject using any suitable fixing device, such as adhesive, pins, etc. Preferably the stereotactic base ring is fixed in position using a series of threaded pins that engage the individual's skull. This ensures the stereotactic base ring is firmly anchored to the subject and will not move during use.

The invention also encompasses the use of the RF coil assembly to obtain a magnetic resonance image of a patient's brain. A magnetic resonance image obtained by using the RF coil assembly may also be provided.

According to a second aspect of the invention, a method of attaching a stereotactic base ring to a subject's head comprises the steps of: (i) taking attachment apparatus, (ii) fixing said attachment apparatus to a stereotactic base ring, (iii) placing the attachment apparatus attached to the stereotactic base ring over the subject's head, and (iv) aligning the stereotactic base ring relative to anatomical features of the subject, characterised in that step (i) comprises taking attachment apparatus according to the first aspect of the invention.

Preferably, step (iv) comprises aligning the stereotactic base ring such that it is substantially parallel to the subjects orbitomeatal plane. Alignment relative to any other plane may be provided if required.

Advantageously, the method comprises the step (v) of fixing the stereotactic frame to the subject's head. Conveniently, step (v) comprises fixing the stereotactic base ring in position using a series of threaded pins.

Preferably, the method comprises the step (vi) of detaching the attachment apparatus from the stereotactic base ring. The attachment apparatus may then be removed from the subject's head.

Once the stereotactic base ring is in place, MRI images may be acquired and/or stereotactic surgery may be performed. Conveniently, the method thus comprises the step (vii) of attaching at least one of an RF coil assembly, a fiducial marker unit, a frame and a stereoguide to the stereotactic base ring. The RF coil assembly may be an RF coil assembly according to the first aspect of the invention.

According to a third aspect of the invention, a method of attaching a stereotactic base ring to an subject's head comprising the steps of: (i) taking attachment apparatus for aligning a stereotactic base ring to a subject's head, (ii) fixing said attachment apparatus to a stereotactic base ring, (iii) placing the attachment apparatus attached to the stereotactic base ring over the subject's head, (iv) aligning the stereotactic base ring relative to anatomical features of the subject, (v) fixing the stereotactic frame to the subject's head, and (vi) detaching the attachment apparatus from the stereotactic base ring. The attachment apparatus of such a method may take the form of the attachment apparatus of the first aspect of the invention.

Conveniently, step (v) comprises fixing the stereotactic base ring in position using a series of threaded pins. Preferably, step (iv) comprises aligning the stereotactic base ring such that it is substantially parallel to the subjects orbitomeatal plane. Conveniently, the method comprises the step (vii) of attaching at least one of an RF coil assembly, a fiducial marker unit, a frame and a stereoguide to the stereotactic base ring. The RF coil assembly may be an RF coil assembly according to the first aspect of the invention.

The present invention thus also provides a method of attaching a stereotactic base ring to an individual's head comprising: fixing the attachment apparatus of the present invention to the stereotactic base ring; placing the attachment apparatus over the individual's head so that it rests on the crown of the individual's head; aligning the stereotactic base ring in a desired plane; and fixing the stereotactic frame in position.

The invention will now be described, by way of example only, with reference to the accompanying drawings, in which:

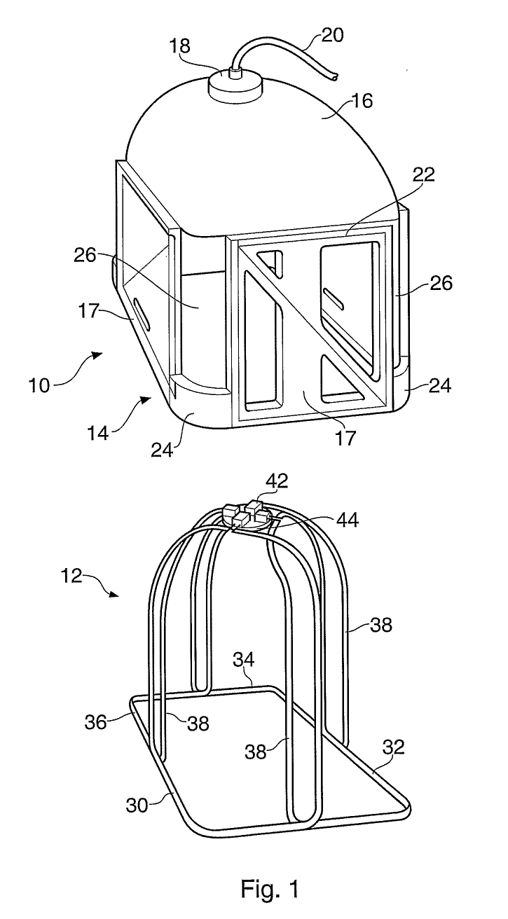

FIG. 1 shows an exploded perspective view of an RF coil assembly and localiser box, wherein the coil structure comprises four coil elements;

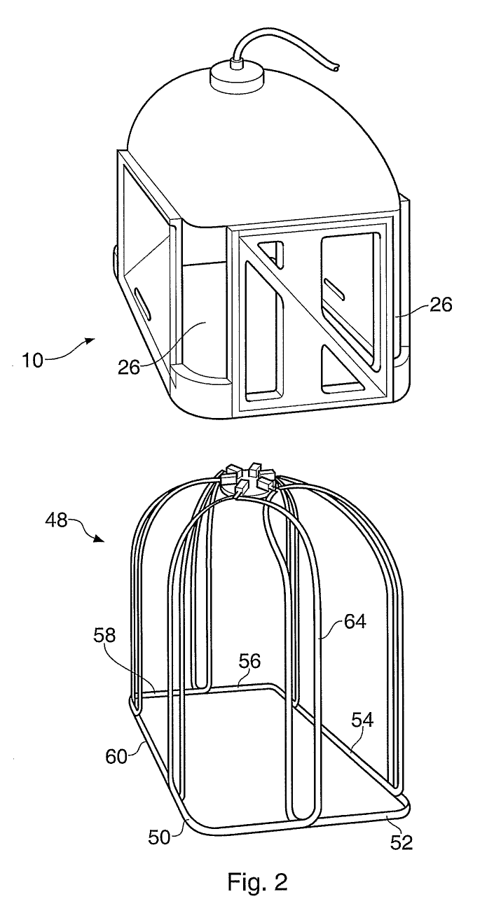

FIG. 2 shows an exploded perspective view of an RF coil assembly and localiser box, wherein the coil structure comprises six coil elements;

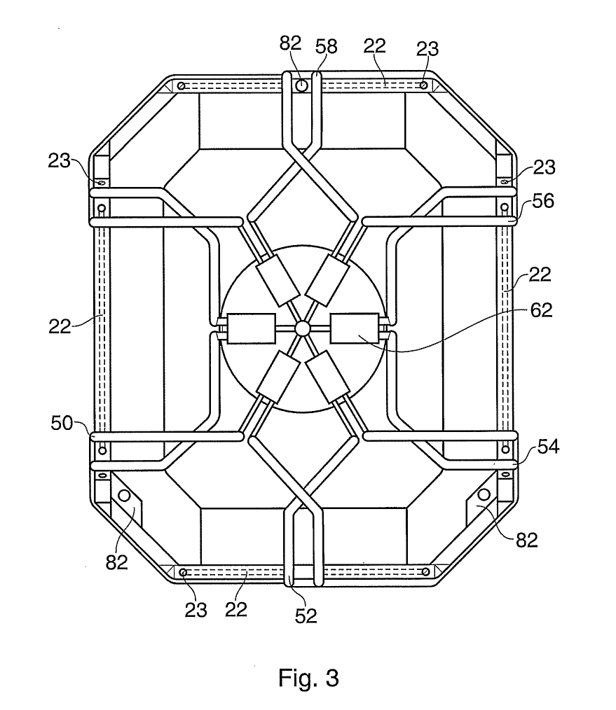

FIG. 3 shows a plan view of an RF coil assembly comprising six coil elements and a stereotactic base ring (mounting details and other features of the base ring are not shown);

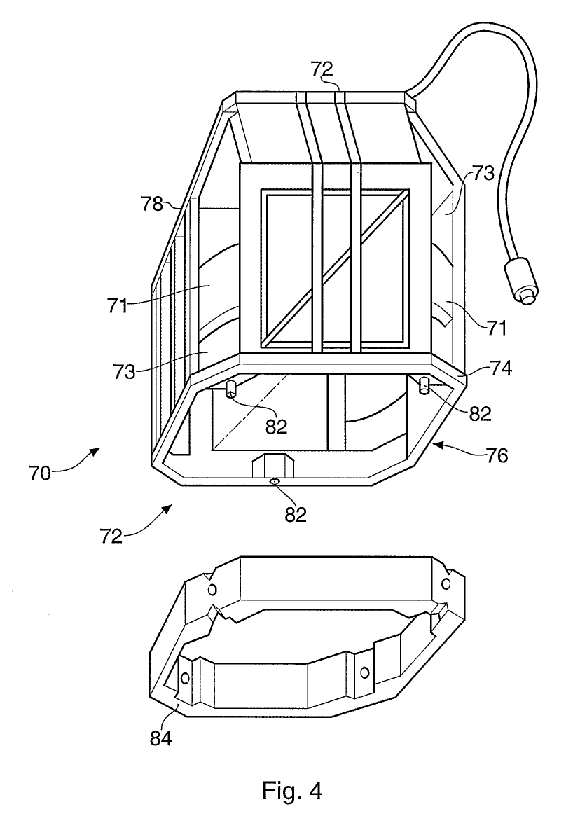

FIG. 4 shows an exploded perspective view of an RF coil assembly and a stereotactic base ring (mounting details and other features of the base ring are not shown), wherein four coil elements are mounted on the outside of the RF coil assembly;

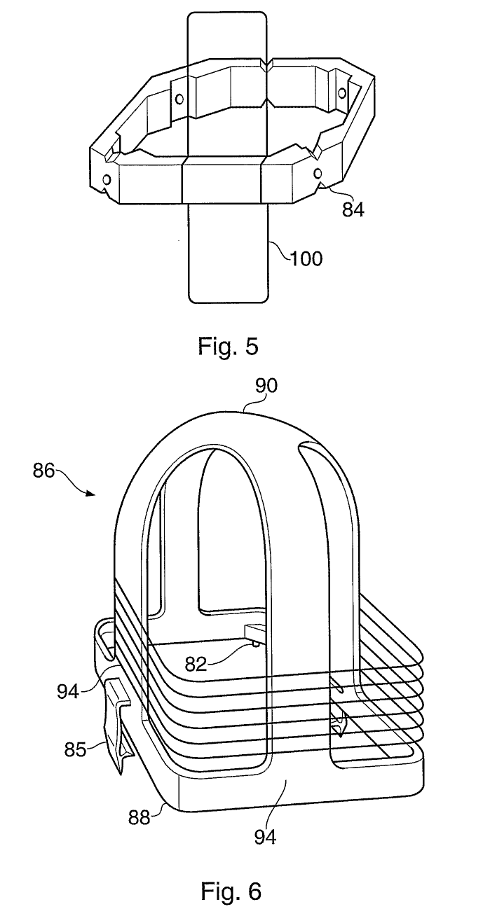

FIG. 5 shows a perspective view of a coil arrangement that allows the coil elements to extend below the stereotactic base ring;

FIG. 6 shows a perspective view of attachment apparatus; and

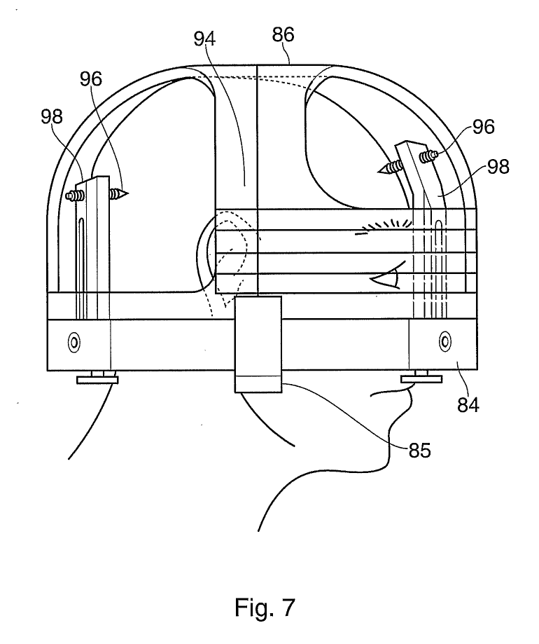

FIG. 7 shows a perspective view of attachment apparatus connected to a stereotactic base ring positioned on an individual's head.

Referring to FIG. 1, an RF coil assembly is shown that has four internal coil elements. The RF coil assembly comprises a localiser box 10 for stereotactic imaging and a receiver coil structure 12 for use with MRI apparatus that comprises four coil elements. The coil structure 12 is formed to fit closely to the inside of the localiser box 10. The coil structure 12 is to be bonded to the inside surface of the localiser box 10 by a thermally conductive adhesive; an RF coil assembly is thus formed that has a coil structure 12 that is integral with the localiser box 10.

The localiser box 10 is open at a first end 14 to allow the localiser box 10 to be placed over a subject's head. A second end 16 opposite to the first end 14 is dome shaped, and has a termination box 18 mounted on it, wherein the termination box 18 is connected to a flexible multicore cable 20 for connection to MRI apparatus. On each of the four sides 17 of the localiser box 10 is mounted a fiducial marker 22. Four collar portions 24 at the first end 14 of the localiser box 10 interconnect the four sides 17. The fiducial markers 22 span substantially the height and width of their respective sides 17 of the localiser box 10. Four open corner edges 26 are defined by the collar portions 24, four sides 17 and second end 16. The localiser box 10 has clips (not shown) for mounting it securely to a standard stereotactic base ring (not shown). The localiser box 10 is made from Perspex and the clips are made from a plastics material. The localiser box 10 has the following dimensions: 240 mm in an anteroposterial axis; 200 mm in a lateral axis; and 150 mm in a vertical axis. The size of the localiser box 10 ensures that it fits closely around an individual's head.

The fiducial markers 22 are shaped to define a square with two opposite corners joined along a diagonal, and are of a standard type for MRI apparatus and comprise oil or copper sulphate solution in tubes.

The coil structure 12 has four preamplifiers 42 located at a first end of the coil structure 12 which are mounted radially on a circular platform 44. The preamplifiers 42 comprise input terminals on outward facing ends and output terminals on inward facing ends. The output terminals of the preamplifiers 42 are connected to a multicore cables which extend to the centre of the platform 44 to form a common feed. When the coil structure 12 is fitted inside the localiser box 10 the preamplifiers 42 are located adjacent the termination box 18 and the common feed is terminated in the termination box 18.

The coil structure 12 is arranged as a phased-array coil system comprising four coil elements 30, 32, 34, 36 where each coil element overlaps with the neighbouring coil element to reduce RF coupling. The coil elements 30, 32, 34, 36 are constructed from a length of 5 mm thick insulated copper ribbon. The coil elements 30, 32, 34, 36 are formed to complement the internal form of the localiser box 10 and are to be attached to the inside surface of the localiser box 10 so that the coil elements 30, 32, 34, 36 are integral with the localiser box 10.

The coil elements 30, 32, 34, 36 are the same size and shape as each other. The portions of the coil elements 30, 32, 34, 36 located within the second end 16 are shaped in the form of an arch and they overlap the neighbouring coil elements. The coil elements each have two longitudinal arms 38 extending down two respective sides 17 of the localiser box 10 to the first end 14. The coils 30, 32, 34, 36 then cross back over the neighbouring coils to form a closed loop which follows the inner circumference of the open end 14. The coil elements 30, 32, 34, 36 connect to the input terminals of their respective preamplifier 42 via radially extending terminals. The coil elements 30, 32, 34, 36 are provided in pairs diametrically opposed from one another. The longitudinal arms 38 of the coil elements are spaced equally around the localiser box 10 and they are located in a mid-portion of the sides 17. The paths of coil elements 30, 32, 34, 36 do not interfere with the open corner edges 26 of the localiser box 10 enabling the posts used to attach the base ring to an individual's head to be accommodated.

The coil elements 30, 32, 34, 36 are provided to have a length such that they have a penetration depth that will incorporate the central volume within the localizer measuring 80 mm laterally, 40 mm antero-posteriorly and 50 mm vertically, and specifically so that there is a common region in the centre of the receiver coil structure 12 from which at least opposite coils can detect electromagnetic energy. The coil elements 30, 32, 34, 36 are capable of being separately tuned and matched at room temperature.

Referring to FIGS. 2 and 3, an RF coil assembly is shown, prior to assembly, that comprises a localiser box 10 and a coil structure 48. The coil structure comprises six coil elements 50, 52, 54, 56, 58, 60 and six preamplifiers 62. The six coil elements 50, 52, 54, 56, 58, 60 are arranged in three pairs of diametrically opposing coil elements and the longitudinal arms 64 are spaced equally around the localiser box 10. Again, as described with reference to FIG. 1, the coils 50, 52, 54, 56, 58, 60 do not interfere with the open corner edges 26 of the localiser box 10. FIG. 3 provides an enlarged view of the top of the coil structure 48 showing the arrangement of the six coils 50, 52, 54, 56, 58, 60 and the six preamplifiers 62. The filling port 23 of the fiducial system 22 and localizer pins 82 for attaching the localiser box 10 to a base ring are also shown.

Referring to FIG. 4 a further example of an RF coil assembly 70 is illustrated. The RF coil assembly includes a phased array coil structure comprising four coil elements 72, 74, 76, 78 that are bonded with adhesive onto the outside of a localiser box. In this manner, RF coil assembly comprises coil elements 72, 74, 76, 78 that are integral with the localiser box.

The RF coil assembly 70 has one open end 79 enabling it to be inserted over an subject's head, and an irregular octagonal cross section with corner portions that are of shorter length than the side portions. The RF coil assembly 70 is provided with linking portions 71 spanning the open corner edges 73. The localiser box portion of the RF coil assembly 70 is constructed from Perspex and has the following dimensions: 240 mm in an anteroposterial axis; 200 mm in a lateral axis; and 150 mm in a vertical axis. The size of the RF coil assembly 70 ensures that it fits closely around an individual's head.

At the open end 79 of the RF coil assembly 70, three pins 82 are provided to attach the RF coil assembly 70 to a stereotactic base ring 84 of a stereotactic frame via corresponding portions on the base ring. The base ring 84 has two v-grooves and a flat portion located in positions corresponding to the pins 82; this provides a kinematic mount that accommodates any distortion of the base ring. Although the base ring 84 comprises two v-grooves and a flat, it may comprise other types of non-kinematic or kinematic mountings (e.g. three v-grooves or holes may be provided on the base ring). The stereotactic base ring 84 is of a standard type capable of being securely attached to an MRI couch by a standard base ring holder.

Referring to FIG. 5, a further RF coil assembly is shown having a coil element 100 that extends below the stereotactic base ring. In particular, a coil 100 is shown that is routed around the outside of the stereotactic base ring 84 and extends below it. Although the coil 100 shown is curved so that it passes around the outside of the stereotactic base ring 84, the coil element could alternatively be routed around the inside of the stereotactic frame 84.

It should be remembered that the above examples are illustrative only. The coil elements need not be arranged in a phased-array coil system, and any number of coil elements could be used in any arrangement suitable for imaging the brain. It would also be apparent to a person skilled in the art that receiver coil elements of the phased array can have a different orientation with respect to each other than that described above and that the phased array may be redesigned to have non-overlapping coil elements.

The above examples also illustrate apparatus in which coil elements are combined with a localiser box. As noted above, a localiser box comprises at least one fiducial marker. The inclusion of such a localiser box is, however, not essential. Instead, a coil support structure (e.g. a plastic box) could be provided which does not include the fiducial markers.

Referring now to FIG. 6, attachment apparatus 86 is shown that comprises a first open end 88 and a second closed end 90. At the first open end 88 there are positioned two clips 85 and localizer pins 82 for attaching the attachment apparatus 86 to a stereotactic base ring. The attachment apparatus 86 also comprises a plurality of markers (a series of parallel, horizontal lines marked 10 mm apart, as well as vertical lines defining the midpoint of the frame in the antero-posterior and lateral axis) 94 to assist in orientating the attachment apparatus 86 with anatomical features of an subject's head. The attachment apparatus 86 is made from Perspex.

The attachment apparatus 86 has substantially the same dimensions as the internal dimensions of the localiser box described above with reference to FIGS. 1 to 4 but does not include fiducial markers 22 or any coil elements. The attachment apparatus 86 also has cut away corners allowing greater access to the individual's head. It is particularly preferred that the attachment apparatus 86 is about 1 to 5 mm shorter than the internal length along the vertical axis (from the open to the closed end) of the associated RF coil assembly to ensure that on attaching the RF coil assembly to a base ring that has been positioned using the attachment apparatus 86, a small gap (about 1 to 5 mm) exists between the crown of the individual's head and the closed end 16 of the RF coil assembly. In this example, the RF coil assembly is as described above with reference to FIG. 1 and therefore the attachment apparatus 86 is sized to position the stereotactic base ring 148 mm away, along the vertical axis, from the crown of the head.

Referring to FIG. 7, the apparatus is shown when located on a subject's head. The attachment apparatus 86 is fixed to the base ring 84 using the clips 85 and location pins 82, and placed over an individual's head so that it rests on the crown of the head. The base ring 84, which is now supported with respect to the individual can be aligned so that it is parallel to the orbitomeatal, or other desirable plane. This is assisted by the series of parallel, horizontal lines 94 marked 10 mm apart on the attachment apparatus which can be aligned with the external auditory meati and inferior orbital margins, and the vertical lines 94 marked on the attachment apparatus defining the midpoint of the attachment apparatus 86 in the antero-posterior and lateral axis.

When appropriately aligned, e.g., to the orbitomeatal line, the base ring 84 is fixed to the head by driving threaded pins 96 positioned on posts 98 extending from the stereotactic base ring 84 into the outer table of the skull. The attachment apparatus 86 is then removed from the stereotactic base ring 84 and replaced with an RF coil assembly of the type described above with reference to FIG. 1. MR imaging can then be performed and the coils of the RF coil assembly are positioned closely to the individual's head thereby maximising image resolution.

The described method of aligning a stereotactic base ring 84 using attachment apparatus prior to stereotactic base ring fixation has advantages over the conventional method which involves inserting ear bars that are attached to the base ring into the patient's external auditory meati and then pivoting the base ring into the desired alignment. In awake patients this procedure can be painful and it is not uncommon for the ear bars to traumatise the external auditory meati and/or drive wax deep into the patients ear and impair the patients hearing.

Again, it should be remembered that the above example of attachment apparatus is illustrative only. Numerous variants of the attachment apparatus may be designed to implement the invention.

1. An RF coil assembly for use with magnetic resonance imaging apparatus, the RF coil assembly comprising one or more coil elements capable of receiving electromagnetic radiation, wherein the RF coil assembly is capable of being directly attached to a stereotactic base ring.

2. An RF coil assembly according to claim 1 comprising at least one coil element capable of transmitting electromagnetic radiation.

3. An RF coil assembly according to claim 1 comprising a coil support structure to which said one or more coil elements are attached, wherein said coil support structure is capable of being directly attached to a stereotactic base ring.

4. An RF coil assembly according to claim 3 wherein the one or more coil elements are rigidly fixed to the coil support structure.

5. An RF coil assembly according to claim 1 comprising a fiducial marker unit.

6. An RF coil assembly according to claim 5 further comprising a coil support structure to which said one or more coil elements are attached, wherein said coil support structure is capable of being directly attached to a stereotactic base ring and wherein the coil support structure incorporates the fiducial marker unit.

7. An RF coil assembly according to claim 5 having a removably attachable fiducial marker unit.

8. An RF coil assembly according to claim 5 wherein the fiducial marker unit encloses said one or more coil elements.

9. An RF coil assembly according to claim 1 wherein the one or more coil elements of the RF coil assembly comprise copper.

10. An RF coil assembly according to claim 1 comprising fixing elements, wherein the fixing elements allow the RF coil assembly to be releasably attached to a stereotactic base ring.

11. An RF coil assembly according to claim 10 wherein said fixing elements allow repeatable relative positioning of the RF coil assembly and the stereotactic base ring.

12. An RF coil assembly according to claim 1 comprising a plurality of coil elements.

13. Attachment apparatus for optimising the attachment position of a stereotactic base ring to a subject's head, the attachment apparatus comprising one or more fixing elements, said one or more fixing elements allowing the attachment apparatus to be releasably attached to a stereotactic base ring, wherein the internal dimensions of the attachment apparatus are substantially the same as the internal dimensions of the RF coil assembly according to claim 1.

14. An attachment apparatus according to claim 13 wherein said apparatus is in the form of a helmet which, in use, contacts the crown of the head and extends, in use, down the sides of the head.

15. An attachment apparatus according to claim 13 comprising one or more markers to enable said attachment apparatus to be aligned with anatomical features of the head.

16. An attachment apparatus according to claim 15 wherein said one or more markers are in the form of one or more reference lines.

17. An attachment apparatus according to claim 15 wherein said one or more markers are located on, or formed integrally with, the attachment apparatus.

18. An attachment apparatus according to claim 15 wherein said markers allow, in use, alignment of the stereotactic base ring with at least one of the external auditory meati, inferior orbital margins and the antero-posterior and lateral axes of the subject.

19. An attachment apparatus according to claim 13 which is made from a substantially transparent material.

20. A kit comprising an RF coil assembly according to claim 1 and an attachment apparatus for optimising the attachment position of a stereotactic base ring to a subject's head, the attachment apparatus comprising one or more fixing elements, said one or more fixing elements allowing the attachment apparatus to be releasably attached to a stereotactic base ring substantially the same as the internal dimensions of the RF coil assembly.

21. A kit according to claim 20 comprising at least one of a stereotactic surgical instrument, a frame, a stereotactic base ring and a base ring holder.

22. The kit according to claim 21 comprising a stereotactic base ring, said stereotactic base ring being capable of being immobilised relative to MRI apparatus.

23. A method of attaching a stereotactic base ring to a subject's head comprising the steps of:
(i) taking attachment apparatus,
(ii) fixing said attachment apparatus to a stereotactic base ring,
(iii) placing the attachment apparatus attached to the stereotactic base ring over the subject's head, and
(iv) aligning the stereotactic base ring relative to anatomical features of the subject, characterised in that step (i) comprises taking attachment apparatus according to claim 13.

24. A method according to claim 23 wherein step (iv) comprises aligning the stereotactic base ring such that it is substantially parallel to the subjects orbitomeatal plane.

25. A method according to claim 23 comprising the step (v) of fixing the stereotactic base ring to the subject's head.

26. A method according to claim 25 wherein step (v) comprises fixing the stereotactic base ring in position using a series of threaded pins.

27. A method according to claim 23 comprising the step (vi) of detaching the attachment apparatus from the stereotactic base ring.

28. A method according to claim 27 comprising the step (vii) of attaching at least one of an RF coil element, a fiducial marker unit, a frame and a stereoguide to the stereotactic base ring.

29. A method according to claim 28 wherein step (vii) comprises the step of attaching an RF coil assembly to the stereotactic base ring, wherein the RF coil assembly is an RF coil assembly for use with magnetic resonance imaging apparatus, the RF coil assembly comprising one or more coil elements capable of receiving electromagnetic radiation, wherein the RF coil assembly is capable of being directly attached to a stereotactic base ring.

30. A method of attaching a stereotactic base ring to a subject's head comprising the steps of:
(i) taking attachment apparatus for aligning a stereotactic base ring to a subject's head,
(ii) fixing said attachment apparatus to a stereotactic base ring,
(iii) placing the attachment apparatus attached to the stereotactic base ring over the subject's head,
(iv) aligning the stereotactic base ring relative to anatomical features of the subject,
(v) fixing the stereotactic frame to the subject's head, and
(vi) detaching the attachment apparatus from the stereotactic base ring.

31. A method according to claim 30 wherein step (v) comprises fixing the stereotactic base ring in position using a series of threaded pins.

32. A method according to claim 30 wherein step (iv) comprises aligning the stereotactic base ring such that it is substantially parallel to the subject's orbitomeatal plane.

33. A method according to claim 30 comprising the step (vii) of attaching at least one of an RF coil element, a fiducial marker unit, a frame and a stereoguide to the stereotactic base ring.

34-36. (canceled)
